(19) United States
(12) Patent Application Publication
(10) Pub. No.: US 2019/0053690 A1
(43) Pub. Date: Feb. 21, 2019

(54) ENDOSCOPE

(71) Applicant: Olympus Corporation, Tokyo (JP)

(72) Inventors: Tatsuhiko Suzuki, Tokyo (JP); Reisuke Osada, Tokyo (JP)

(73) Assignee: Olympus Corporation, Tokyo (JP)

(21) Appl. No.: 16/166,414

(22) Filed: Oct. 22, 2018

Related U.S. Application Data
(63) Continuation of application No. PCT/JP2017/006919, filed on Feb. 23, 2017.

(30) Foreign Application Priority Data
Apr. 25, 2016 (JP) 2016-087180

(51) Int. Cl. A61B 1/005; A61B 1/00

(52) U.S. Cl. CPC A61B 1/0052; G02B 23/2476; A61B 1/0055; A61B 1/00066

(57) ABSTRACT

An endoscope includes an insertion portion, a manipulator, a shaft, and a finger rest. The manipulator is disposed on a proximal end side of the insertion portion and having a grip. A shaft is disposed outward from the manipulator and standing up in a neutral direction when not manipulated and configured to be tilted toward at least the insertion portion and the grip. The finger rest is disposed on an end portion of the shaft. The finger rest has an outer surface. The outer surface includes a first surface and a second surface. The first surface is located on the side of the insertion portion with extending about a first central point. The second surface is located on the side of the grip with extending about a second central point. The second central point is positioned closer to the insertion portion than the first central point.

### CROSS-REFERENCE TO RELATED APPLICATIONS

This application is a continuation application of PCT Application No. PCT/JP2017/006919 filed on Feb. 23, 2017, which in turn claim priority to the Japanese Patent Application No. 2016-87180 filed on Apr. 25, 2016 in Japan which is hereby incorporated by reference in its entirety.

### TECHNICAL FIELD

The technology disclosed herein generally relates to an endoscope having a bend manipulating member on a manipulator thereof. The bend manipulating member can be tilted by a finger of a user that grips the manipulator of the endoscope.

### DESCRIPTION OF THE RELATED ART

Endoscopes include a tubular insertion portion with an observational optical system located near its distal end. Some endoscopes have a bendable portion on the distal end side of an insertion portion. The bendable portion can be bent in two directions, i.e., upward and downward directions, or in four directions, i.e., upward, downward, leftward, and rightward directions. The endoscopes with the bendable portion on the insertion portion allow the insertion portion to be inserted easily into a body for diagnostic purposes. Also, the endoscopes can change the visual field direction for observation in a wide range.

Generally, a manipulator is disposed on the proximal end of the insertion portion and is used as a grip. The manipulator includes a bend manipulating device for bending the bendable portion. The bend manipulating device of the manipulator can be manipulated by a hand finger of the user, such as the user, a doctor or the like, who grips the grip. An endoscope includes a rod-shaped bend manipulating member as the bend manipulating device. When the user tilts the bend manipulating member through a desired angle in a desired direction, a bending wire is pulled and loosened so as to bend the bendable portion to a desired extent.

Japanese Patent No. 5238099, for example, discloses a medical device with a bendable portion. The medical device includes a manipulating element used as a bend manipulating member with excellent operability. The manipulating element extends perpendicularly to a gripping surface of a manipulator. The manipulating element can stably be manipulated to make arcuate motion and can be tilted in an increased range without letting the finger slip off. In the medical device with a bendable portion, a finger contact, which corresponds to a finger rest according to the present disclosure, is integrally attached to the distal end of a shaft of the manipulating element. The finger contact is shaped as a pentagonal body having a bottom face, a first operation surface, a second operation surface, a third operation surface, and a fourth operation surface, or shaped as a hexagonal body having a bottom face, a first operation surface, a second operation surface, a third operation surface, a fourth operation surface, and a top face.

For bending the bendable portion, the user places a thumb pad on the third operation surface, the fourth operation surface, or the top face, or places a thumb side on the first operation surface or the second operation surface to tilt the manipulating element. The medical device with a bendable portion is arranged such that the user can bend the bendable portion to a large extent by placing a thumb tip on one side of the bottom face of the finger contact and tilting the finger contact. The size of a hand of the user that manipulates the endoscope differs from user to user, and thus the operability of the manipulating element varies with the different sizes of hands used. A user with a smaller hand may tend to fail to have a thumb reach as far as desired in a push-over action when the user tilts the manipulating element in the direction toward the insertion portion with the thumb of the hand that is gripping the grip. On the other hand, a user with a larger hand may tend to have a thumb seize up, failing to tilt the manipulating element smoothly, in a pull-over action when the user pulls the manipulating element over toward the user away from the insertion portion with the thumb of the hand that is gripping the grip.

### BRIEF SUMMARY OF EMBODIMENTS

The technology disclosed herein has been made in view of the above difficulties. It is an object of the present disclosure to provide an endoscope that includes a manipulator having an upstanding bend manipulating member that can smoothly be tilted by a finger of the hand of the user that is gripping the manipulator, placed on an outer surface of a finger contact, regardless of the size of the user's hand or the length of the user's finger.

According to an aspect of the present disclosure, an endoscope includes a bendable portion, a manipulator, a bend manipulating member, and a finger rest. The finger rest has a first surface and a second surface. The bendable portion is disposed in an insertion portion and bendable at least upwardly and downwardly. The manipulator is disposed on a proximal end side of the insertion portion and includes a grip that can be gripped by a user. The bend manipulating member is disposed between the insertion portion and the grip. The bend manipulating member stands up in a predetermined neutral direction when not manipulated. The bend manipulating member is tiltable toward at least the insertion portion and the grip. The finger rest is disposed on an end portion of the bend manipulating member positioned outside of the manipulator. The user can place a thumb thereof on the finger rest while gripping the grip. The first surface is an arcuate and defined as an outer surface of the finger rest on a side of the insertion portion. The first surface extends about a first central point and has a first radius. The second surface is an arcuate and defined as an outer surface of the finger rest on a side of the grip. The second surface extends about a second central point positioned closer to the insertion portion than the first central point and the second surface has a second radius.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view of a portion of an endoscope, primarily illustrating a manipulator and a bend manipulating member thereof.

FIG. 2 is a view illustrating examples of arcuate surfaces on an outer surface of a finger rest.

FIG. 3A is a view illustrating an outer surface of a finger rest attached to a shaft according to an embodiment of the present disclosure.

FIG. 3B is a view illustrating a push-over action.

FIG. 3C is a view illustrating a pull-over action.

FIG. 4 is a view illustrating the manner in which the central axis of the shaft is tilted through an angle larger than 90 degrees with respect to a pedestal mount flat surface.

FIG. 5 is a view illustrating a plurality of bumps disposed on the outer surface of the finger rest.

FIG. 6A is a front elevational view of the outer surface of the finger rest, illustrating an example of a layout of bumps disposed on the outer surface of the finger rest.

FIG. 6B is a front elevational view of the outer surface of another finger rest, illustrating another example of a layout of bumps disposed on the outer surface of the finger rest.

FIG. 7A is a front elevational view of the outer surface of still another finger rest, illustrating still another example of a layout of bumps disposed on the outer surface of the finger rest.

FIG. 7B is a view of the finger rest depicted in FIG. 7A, as viewed from an insertion portion of the endoscope.

FIG. 7C is a left-hand side elevational view of the finger rest depicted in FIG. 7A.

FIG. 8A is a front elevational view of the outer surface of yet another finger rest, illustrating yet another example of a layout of bumps disposed on the outer surface of the finger rest.

FIG. 8B is a view of the finger rest depicted in FIG. 8A, as viewed from the insertion portion of the endoscope.

FIG. 8C is a left-hand side elevational view of the finger rest depicted in FIG. 8A.

FIG. 9A is a front elevational view of the outer surface of yet still another finger rest, illustrating yet still another example of a layout of bumps disposed on the outer surface of the finger rest.

FIG. 9B is a view of the finger rest depicted in FIG. 9A, as viewed from the insertion portion of the endoscope.

FIG. 9C is a left-hand side elevational view of the finger rest depicted in FIG. 9A.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

The figures referred to in the description hereinafter are schematically illustrative of the present disclosure, and may depict members at different dimensions, scales, etc. for components in order to illustrate the components to the extent that they are recognizable in the figures. Consequently, the present invention is not limited only to the illustrated forms in terms of the numbers of the components illustrated in the figures, the shapes of the components, the ratios of the sizes of the components, the relative positional relationships between the components, and so on.

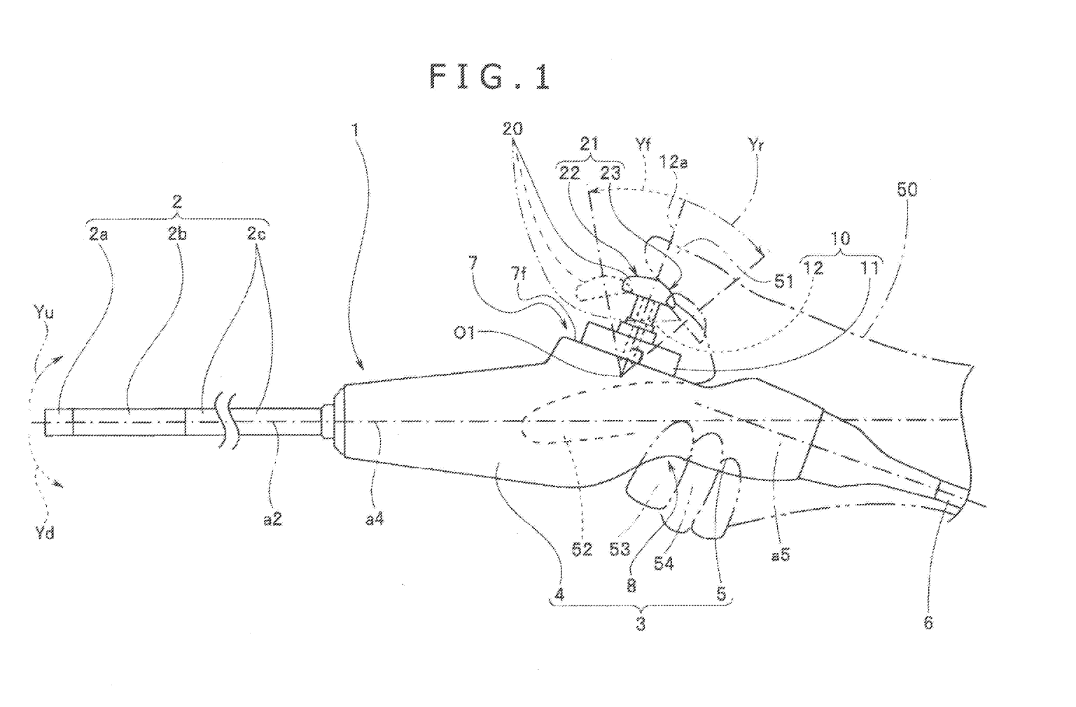

As depicted in FIG. 1, an endoscope 1 has a tubular insertion portion 2 and a manipulator 3. The insertion portion 2 is insertable into a body of an examinee. The insertion portion 2 includes a distal-end portion 2a, a bendable portion 2b, and a rigid pipe 2c that are successively arranged from the distal end thereof. The distal-end portion 2a contains therein an image capturing unit having an image capturing device such as a CCD, a CMOS circuit, or the like. The bendable portion 2b is bendable in four directions, i.e., upward, downward, leftward, and rightward directions, for example. The rigid pipe 2c is a pipe of stainless steel or a pipe of hard synthetic resin. The bendable portion 2b may be bendable in two directions, i.e., upward and downward directions. The insertion portion 2 may include a flexible pipe that is pliable and flexible, instead of the rigid pipe 2c.

The manipulator 3 has a tubular manipulator main body 4 and a grip 5. The manipulator main body 4 is positioned on a proximal end side of the insertion portion 2, whereas the grip 5 is positioned on a proximal end side of the manipulator main body 4. A universal cord 6 extends from a proximal end side of the grip 5. The manipulator main body 4 has a longitudinal axis a4 and the insertion portion 2 has a longitudinal axis a2, the longitudinal axis a4 and the longitudinal axis a2 lying coplanar to each other. The grip 5 has a longitudinal axis a5 crossing the longitudinal axis a4 of the manipulator main body 4. In other words, the manipulator 3 has the manipulator main body 4 and the grip 5, and is oriented in a bent shape. A pedestal 7 and a finger hooking recess 8 are disposed at predetermined positions on an intermediate region of the manipulator 3. The user grips the manipulator 3 with an illustrated hand 50. The finger hooking recess 8 is a cavity where a middle finger 53 of the hand 50 is placed as indicated by the two-dot-and-dash lines. Not only the middle finger 53, but also an index finger 52 and a ring finger 54 can be placed in the finger hooking recess 8. The index finger 52 is indicated by the broken lines in FIG. 1. Though the right hand 50 is illustrated as gripping the manipulator 3, the left hand may grip the manipulator 3.

The pedestal 7 is disposed across the longitudinal axis a4 from the finger hooking recess 8. The pedestal 7 has a pedestal mount flat surface 7f. According to the present embodiment, the pedestal mount flat surface 7f lies substantially parallel to the longitudinal axis a5 of the grip 5. The pedestal mount flat surface 7f is not limited to lying parallel to the longitudinal axis a5 of the grip 5, but should preferably be inclined to the longitudinal axis a4 of the manipulator main body 4. However, the pedestal mount flat surface 7f may be a surface parallel to the longitudinal axis a4 of the manipulator main body 4. A cover 11 and a shaft 12 make up a bend manipulating device 10 and project outwardly from the pedestal mount flat surface 7f. The cover 11 is an elastic member having a predetermined shape with a predetermined elastic force. The cover 11 hermetically closes an opening, not depicted, defined in the pedestal mount flat surface 7f. The opening leads to a space in the manipulator main body 4. The shaft 12 projects outwardly through a hole defined in the cover 11. A finger rest 20 is attached to the end of the shaft 12 that projects outwardly.

The shaft 12 is used as a bend manipulating member for bending the bendable portion 2b by directly pulling a bend manipulating wire, not depicted. According to the present embodiment, the shaft 12 has a central axis 12a (i) standing up perpendicularly to the longitudinal axis a5 of the grip 5 and (ii) erected from the pedestal mount flat surface 7f. The central axis 12a thus oriented is in a neutral direction. When the shaft 12 stands up in the neutral direction, the bendable portion 2b is in a substantially straight state. The shaft 12 that stands up in the neutral direction is tiltable about a center O1. Specifically, the shaft 12 is tiltable through 30 degrees, for example, in all directions by an illustrated thumb 51 indicated by the two-dot-and-dash lines. The thumb 51 of the hand 50 of the user is placed on a manipulator outer surface (hereinafter referred to as “outer surface”) 21. The outer surface is a manipulating surface of the finger rest 20. When the thumb 51 tilts the finger rest 20 from the neutral direction toward the insertion portion 2 as indicated by the broken-line arrow Yf in FIG. 1 and pushes over the shaft 12, the bendable portion 2b is bent to move the distal-end portion 2a downwardly as indicated by the broken-line arrow Yd.

In contrast, when the thumb 51 tilts the finger rest 20 from the neutral direction toward the grip 5 as indicated by the two-dot-and-dash-line arrow Yr in FIG. 1 and pulls over the shaft 12, the bendable portion 2b is bent to move the distal-end portion 2a upwardly as indicated by the two-dot-and-dash-line arrow Yu. Conversely, the endoscope 1 may be arranged such that when the shaft 12 is pushed over toward the insertion portion 2, the distal-end portion 2a moves upwardly, and when the shaft 12 is pulled over toward the grip 5, the distal-end portion 2a moves downwardly. The endoscope 1 is arranged such that when the shaft 12 is tilted to the right, the bendable portion 2b of the insertion portion 2 is bent to move the distal-end portion 2a to the right, and when the shaft 12 is tilted to the left, the bendable portion 2b of the insertion portion 2 is bent to move the distal-end portion 2a to the left. The thumb pad of the user or operator is placed on the outer surface 21 of the finger rest 20. According to the present embodiment, the outer surface 21 of the finger rest 20 is configured to allow a user with large hands and a user with small hands to tilt the shaft 12 smoothly and reliably.

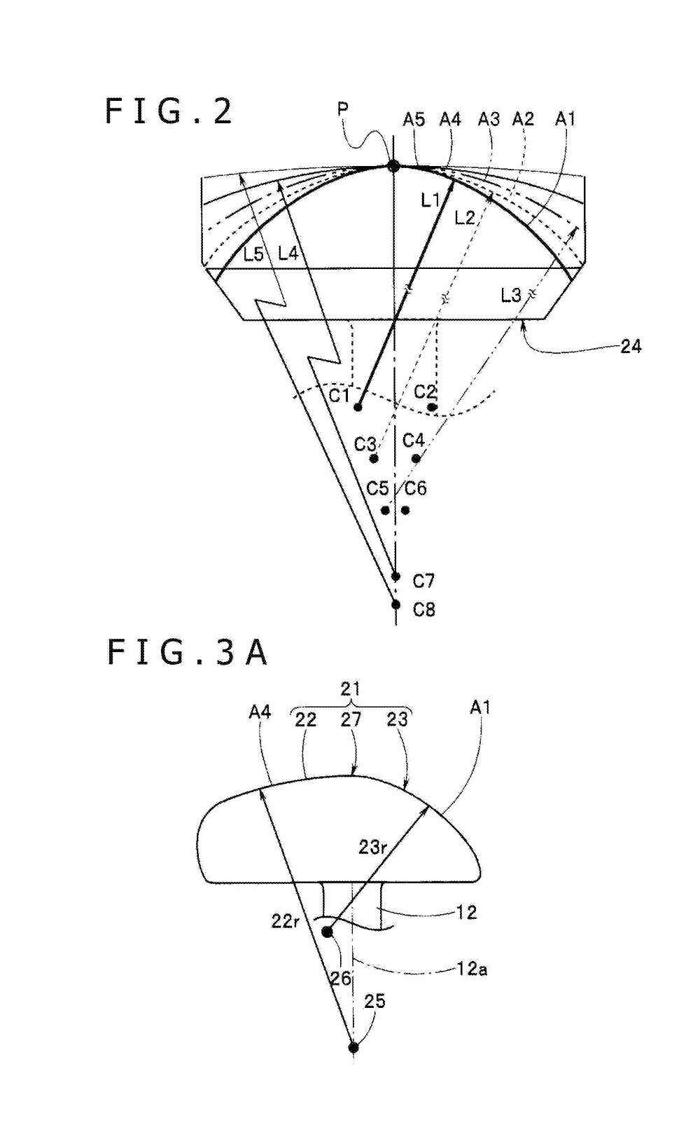

Specifically, the outer surface 21 of the finger rest 20 has a first surface 22 and a second surface 23. The first surface 22 is disposed on the side of insertion portion 2. The first surface 22 can be said as an insert-side finger rest face. The second surface 23 can be said as a grip-side finger rest face. The second surface 23 is disposed on the side of grip 5. The respective first surface 22 and the second surface 23 include respective arcuate faces that have different surface shapes in view of the operability to tilt the finger rest 20. The shape of the first and second surface 22 23 are set as follows. First, a finger rest 20 having a first arcuate face A1 that is an arcuate surface indicated by the thick solid line in FIG. 2 is formed. Then, a finger rest 20 having a second arcuate face A2 indicated by the broken lines is formed. Then, a finger rest 20 having a third arcuate face A3 indicated by the two-dot-and-dash lines is formed. Then, a finger rest 20 having a fourth arcuate face A4 indicated by the solid line that is thinner than the thick solid line representing the first arcuate face A1 is formed. Then, a finger rest 20 having a fifth arcuate face A5 indicated by the solid line that is thinner than the solid line representing the fourth arcuate face A4 is formed.

The first arcuate face A1 is represented by (i) an arc extending about a first central position C1 and having a radius represented by a first length L1 and (ii) an arc extending about a second central position C2 disposed in symmetric relation to the first central position C1 across the central axis 12a and having a radius represented by the first length L1. The second arcuate face A2 is represented by two arcs each extending about (i) a third central position C3 that is different from the first central position C1 and the second central position C2 and (ii) a fourth central position C4 disposed in symmetric relation to the third central position C3 across the central axis 12a, and having respective radii each represented by a second length L2 larger than the first length L1. The third arcuate face A3 is represented by two arcs each extending about (i) a fifth central position C5 that is different from the central positions C1, C2, C3, and C4 and (ii) a sixth central position C6 disposed in symmetric relation to the fifth central position C5 across the central axis 12a, and having respective radii each represented by a third length L3 larger than the second length L2. The fourth arcuate face A4 is, for example, represented by an arc extending about a seventh central position C7 on the central axis 12a and having a radius represented by a fourth length L4 larger than the third length L3. The fifth arcuate face A5 is represented by an arc extending about an eighth central position C8 on the central axis 12a and having a radius represented by a fifth length L5 larger than the fourth length L4.

A vertex “P” is located at a highest position from a bottom face 24 and is positioned in the vicinity of the central axis 12a. These multiple arcuate faces are formed freely by setting appropriate central positions and lengths serving as radii. The arcuate faces are not limited to the five kinds described hereinbefore, but may be of more kinds. The operability of the respective finger rests 20 with the multiple arcuate faces will be verified below.

Specifically, the finger rest 20 with the first arcuate face A1 as the first surface 22 and the second surface 23 is attachably mounted on the shaft 12, and the operability of the finger rest 20 is verified in push-over and pull-over actions made by a user with large hands, a person with small hands, and a person with general-size hands. Similarly, the finger rest 20 with the second arcuate face A2 as the first surface 22 and the second surface 23 is attachably mounted on the shaft 12, and the operability of the finger rest 20 is verified by users having differently sized hands in the same manner as described hereinbefore. Furthermore, (i) the operability of the finger rest 20 with the third arcuate face A3 as the first surface 22 and the second surface 23, (ii) the finger rest 20 with the fourth arcuate face A4 as the first surface 22 and the second surface 23, and (iii) the finger rest 20 with the fifth arcuate face A5 as the first surface 22 and the second surface 23, are verified by users having differently sized hands in the same manner as described hereinbefore.

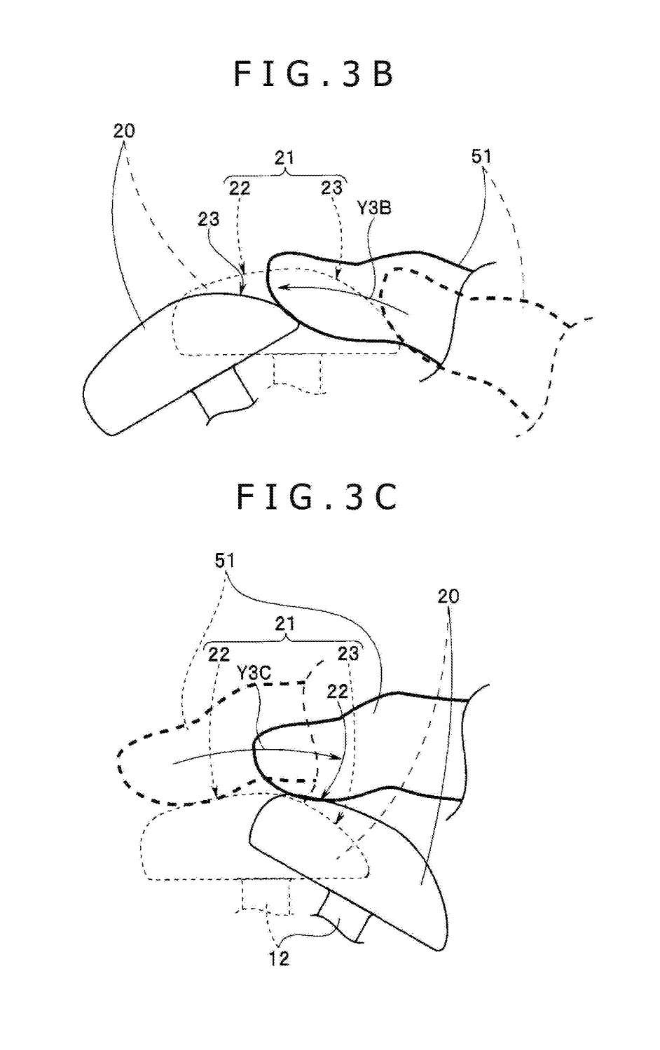

After the verification, it has been confirmed that of the first through fifth arcuate faces A1 through A5, the fourth arcuate face A4 is suitable for a pull-over action made by the both users with the large hands and with the small hands. It has also been confirmed that of the first through fifth arcuate faces A1 through A5, the first arcuate face A1 is suitable for a push-over action made by the both users with the small hands and with the large hands. In the pull-over action made by the user or operator with the small hands, (i) the fifth arcuate face A5 is likely to let the thumb slip as it is of a smaller curvature than the fourth arcuate face A4, and (ii) the third arcuate face A3 is difficult to reach by the finger of the user with the small hands as it is of a larger curvature than the fourth arcuate face A4. The outer surface 21 of the finger rest 20 is configured as depicted in FIG. 3A based on the above results of the verification. Specifically, the first surface 22 of the outer surface 21 is made up of the fourth arcuate face A4 depicted in FIG. 2 and the second surface 23 thereof is made up of the first arcuate face A1 depicted in FIG. 2.

Stated otherwise, the outer surface 21 of the finger rest 20 has the first surface 22 and the second surface 23 which are represented by different arcuate faces. The first surface 22 is, for example, represented by an arcuate face extending about a first central point 25 at the seventh central position C7 depicted in FIG. 2 and having a first radius 22r represented by the length L4. The second surface 23 is represented by an arcuate face extending about a second central point 26 at the first central position C1 depicted in FIG. 2 and having a second radius 23r represented by the length L1. The first central position C1 is positioned closer to the insertion portion 2 side than the first central point 25. A junction 27 is a junction where the proximal-end side of the first surface 22 and the distal-end side of the second surface 23 are joined. The junction is made up of a vertex, a curved surface including a vertex, or a flat surface including a vertex.

In this manner, the outer surface 21 of the finger rest 20 is made up of the first surface 22 and the second surface 23 that are represented by arcuate faces extending about different central points and having different radii. The second central point 26 of the second surface 23 is positioned closer to the insertion portion 2 side than the first central point 25 of the first surface 22, and the first radius 22r of the first surface 22 is larger than the second radius 23r of the second surface 23. There is thus provided the finger rest 20 having the outer surface 21 that includes the first surface 22 suitable for a pull-over action made by both users with large hands and with small hands, and the second surface 23 suitable for a push-over action made by users with small hands and with large hands.

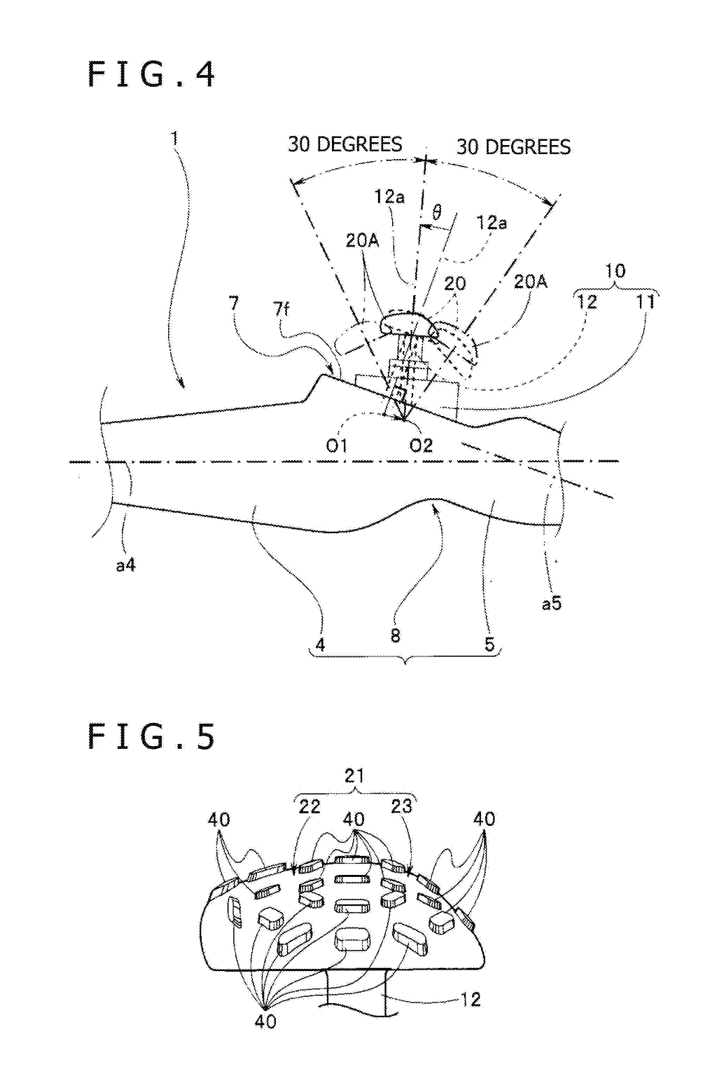

In other words, the user places the thumb 51 of the hand 50 that grips the manipulator 3 on the outer surface 21 of the finger rest 20 as depicted in FIG. 1, and can smoothly and reliably perform tilting actions including a push-over action indicated by the arrow Y3B in FIG. 3B and a pull-over action indicated by the arrow Y3C in FIG. 3C, regardless of the size of the hand 50 of the user. In the above embodiment, the central axis 12a of the shaft 12 extends perpendicularly to the pedestal mount flat surface 7f. However, the central axis 12a of the shaft 12 may be inclined to and extend across the pedestal mount flat surface 7f at an angle of 105 degrees (in FIG. 4, θ=15 degrees), for example, larger than 90 degrees. The shaft 12 thus inclined at 105 degrees has a center O2 positionally offset from the center O1 so that a user with small hands can place the thumb 51 on a finger rest 20A on the shaft 12 with a middle finger 53 placed in the finger hooking recess 8, and can tilt the finger rest 20A toward the insertion portion 2 and the grip 5 as indicated by the two-dot-and-dash lines.

In the present embodiment, the central axis 12a of the shaft 12 at the time it is inclined 105 degrees to the pedestal mount flat surface 7f is in the neutral direction. The shaft 12 that stands up in the neutral direction is tiltable about the center O2 through 30 degrees, for example, in all directions. Other structural details are identical to those of the embodiment described hereinbefore, and identical parts are denoted by identical reference symbols and will not be described in detail hereinafter. As the angle at which the central axis 12a of the shaft 12 crosses the pedestal mount flat surface 7f is set to an angle of 105 degrees, for example, larger than 90 degrees, when the finger rest 20A is pulled over toward the grip 5, the position of the finger rest 20A is changed to a position indicated by the two-dot-and-dash lines which is positionally offset upwardly from the position of the finger rest 20 indicated by the broken lines. As a result, operability in a push-over action and a pull-over action made by a user with small hands are not changed, and a pull-over action made by a user with large hands can be performed more smoothly. Other operational details and advantages are similar to those of the embodiment described hereinbefore.

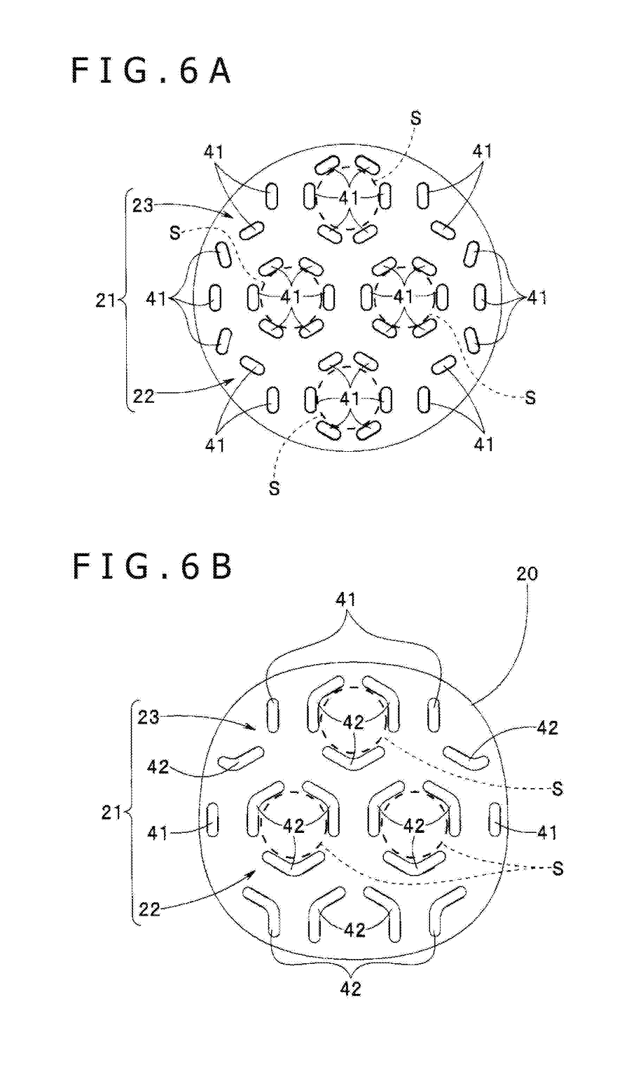

The angle at which the central axis 12a of the shaft 12 crosses the pedestal mount flat surface 7f is 105 degrees. However, the angle at which the central axis 12a crosses the pedestal mount flat surface 7f is not limited to 105 degrees larger than 90 degrees, but may be set to a value in the range of 90 to 130 degrees for obtaining the advantages described hereinbefore. As depicted in FIG. 5, the first surface 22 and the second surface 23 which are arcuate faces of the finger rest 20 may have a plurality of bumps 40 thereon for preventing a finger of a surgeon who is wearing medical gloves from slipping.

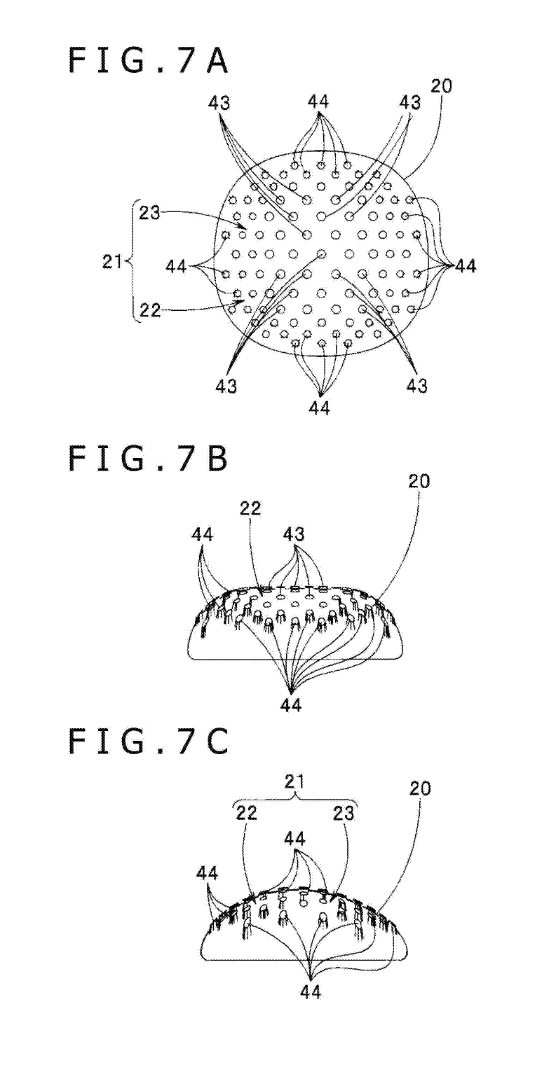

In FIG. 6A, a plurality of bumps 41 are columnar bumps arranged in groups of six bumps 41 on the outer surface 21 that are arrayed at predetermined intervals in a hexagonal shape. A hexagonal shape cannot be formed in empty spaces such as a left side surface, a right side surface, and so on. Therefore, columnar bumps are arrayed at predetermined intervals in a substantially triangular shape. Though the bumps 41 are illustrated as arrayed in hexagonal shapes, they may be arrayed in polygonal shapes other than the hexagonal shapes, or circular shapes or desired shapes such as elliptical shapes or the like. Inner areas “S” indicated by the broken lines are surrounded by the bumps arrayed in hexagonal shapes and are free of bumps, so that the finger pad falls of its own accord into the inner areas “S”. As the bumps 41 are arrayed in predetermined shapes on the first surface 22 and the second surface 23 of the finger rest 20, the finger pad of the finger of the surgeon who is wearing medical gloves falls into the areas “S” surrounded by the bumps 41 and touches the wall surfaces of inner portions or outer portions of the bumps 41, so that the finger is stably caught by the bumps 41. As a consequence, the finger in tilting actions is reliably prevented from slipping off the outer surface 21 for increased tilting operability.

Because the bumps 41 are arrayed at predetermined intervals and arranged in desired shapes, liquid such as water or the like that comes in contact with the finger is easier to flow through the gaps between the bumps 41 than if bumps are arranged in gap-free shapes. As a result, the finger with water applied thereto is prevented from slipping for increased tilting operability. When the endoscope 1 is cleaned, its cleanability is maintained at a high level regardless of the bumps 41 arranged in desired shapes. In case the bumps 41 are arranged in a hexagonal shape, adjacent ones of the bumps 41 on the outer surface 21 are oriented in different directions. As a result, the finger pad is caught by the wall surface of either one of the bumps 41. Therefore, the finger rest 20 reliably prevents the finger of the user or surgeon in tilting actions from slipping off the outer surface 21 regardless of the direction of the finger in the tilting actions.

In FIG. 6B, bumps 42 are substantially columnar bumps. In this case, groups of three bumps 42 are arranged in a hexagonal shape and are disposed on the outer surface 21 so as to prevent a finger from slipping off the outer surface 21. In empty spaces such as a left side surface, a right side surface, and so on where a hexagonal shape cannot be formed, the columnar bumps 41 and the chevron-shaped bumps 42 are appropriately disposed to prevent a finger from slipping off the outer surface 21.

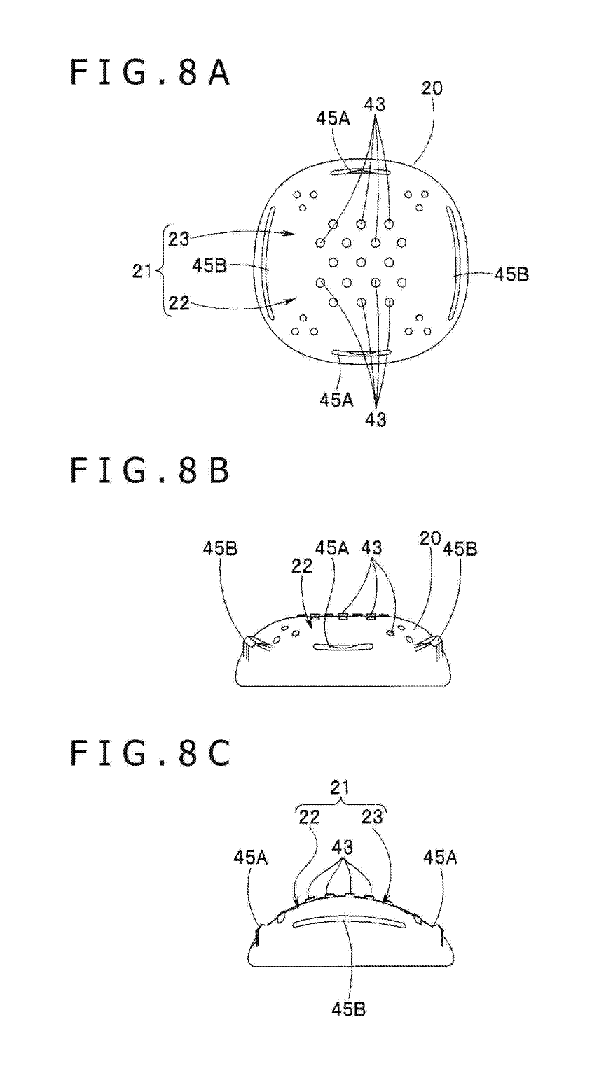

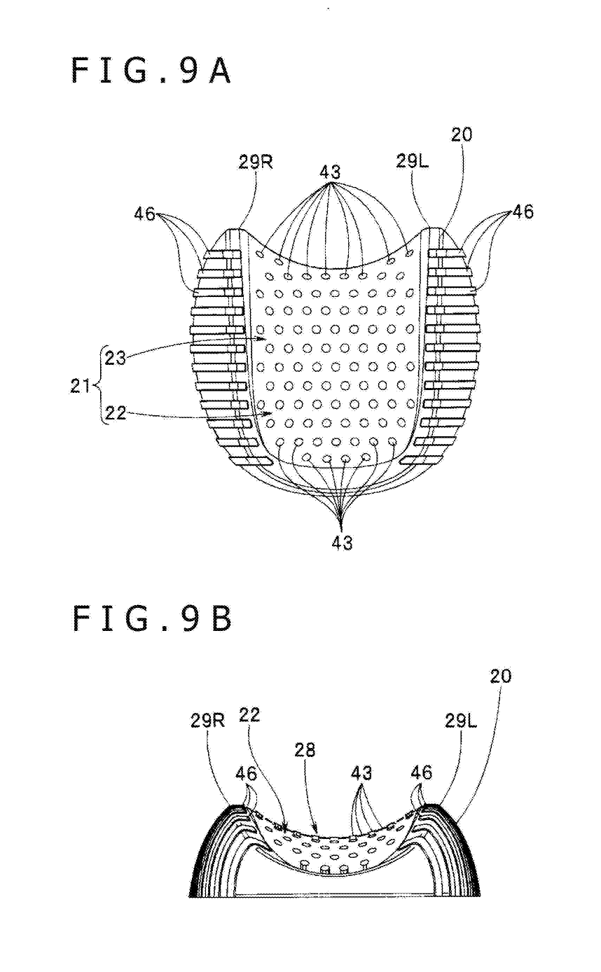

As depicted in FIGS. 6A and 6B, the outer surface 21 of the finger rest 20 as it is viewed in the neutral direction is of a circular shape, a substantially oblong shape, or the like. In the above embodiment, the bumps include columnar bumps 41 or chevron-shaped bumps 42. However, the bumps may include dots 43 and cylindrical bumps 44 as depicted in FIGS. 7A through 7C, or may include dots 43 and circumferential columnar bumps 45A and 45B as depicted in FIGS. 8A through 8C for preventing a finger from slipping off the outer surface 21.

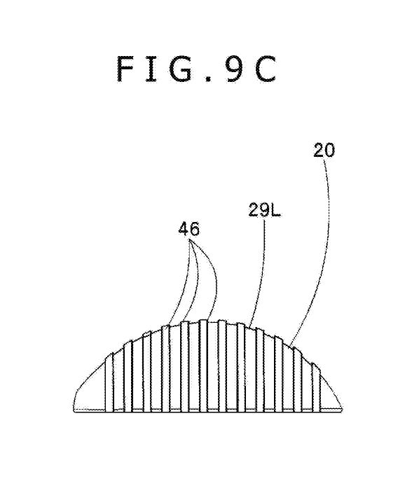

As depicted in FIGS. 7B and 7C, the dots 43 are disposed in a central area of the outer surface 21, and the cylindrical bumps 44 are disposed on an insert side surface, a grip side surface, a left side surface, and a right side surface of the outer surface 21. As depicted in FIGS. 8A through 8C, the first circumferential columnar bumps 45A are disposed on a first surface and a second surface of the outer surface 21. The first surface is on a side of insertion portion and the second surface is on a side of the grip of. The second circumferential columnar bumps 45B are disposed on a left side surface and a right side surface of the outer surface 21. The finger rest 20 is not limited to the circular shape and the substantially oblong shape, but may be of a saddle shape as depicted in FIGS. 9A through 9C.

As depicted in FIGS. 9A and 9B, the finger rest 20 according to the present embodiment has a groove 28 capable of accommodating the pad of a thumb, for example, therein and is of a saddle shape. The groove 28 has a bottom surface that is a manipulating surface and the outer surface 21. The groove 28 includes the first surface 22 and the second surface 23 described hereinbefore. The reference symbol 29L denotes a left side wall, and the reference symbol 29R denotes a right side wall. A plurality of dots 43 are arrayed on the first surface 22 and the second surface 23 for preventing a finger from slipping off the outer surface 21. The left side wall 29L forms a left side surface. A plurality of bumps 46 extends from the upper face to the bottom face 24 on the left side surface. The plurality of bumps 46 are arrayed between the insertion portion side and the grip side. In addition, a right side surface is formed by the right side wall 29R. A plurality of bumps 46 extends from the upper face to the bottom face 24 on the right side surface. The plurality of bumps 46 are arrayed between the insertion portion side and the grip side.

As described hereinbefore, the finger rest 20 has, in addition to the groove 28, the left side wall 29L, and the right side wall 29R, the plurality of dots 43 on the bottom surface of the groove 28 for preventing a finger from slipping off the outer surface 21. As a result, when a finger accommodated in the groove 28 tilts the shaft 12 toward the insertion portion 2 or the grip 5, the finger pad of the finger accommodated in the groove 28 touches the dots 43 on the bottom surface of the groove 28, preventing the finger from slipping off the outer surface 21. In addition, when the finger accommodated in the groove 28 tilts the shaft 12 to the left, the left side wall 29L reliably prevents the finger from slipping off the outer surface 21, and when the finger accommodated in the groove 28 tilts the shaft 12 to the right, the right side wall 29R reliably prevents the finger from slipping off the outer surface 21. Since the finger rest 20 is of a saddle shape having the groove 28, the left side wall 29L, and the right side wall 29R, the finger rest 20 is more effective to prevent a finger from slipping off the upper surface 21.

As the bumps 46 extending from the upper surface to the bottom surface 24 are disposed on the left side surface of the left side wall 29L and the right side surface of the right side wall 29R, the user or operator may place a thumb on the upper surface or the left side surface of the left side wall 29L or the upper surface or the right side surface of the right side wall 29R, rather than in the groove 28, and perform a tiling action. In the above embodiment, the shaft 12 that projects outwardly is tilted to bend the bendable portion 2b by directly pulling the bend manipulating wire. However, the endoscope may incorporate an electric bending mechanism for bending the bendable portion by pulling the bend manipulating wire with a drive force or the like from an electric motor based on (i) the direction in which the shaft 12 is tilted and (ii) the angle through which the shaft 12 is tilted. The finger rest 20 allows the user to tilt the shaft 12 smoothly regardless of the size of the user's hand or the length of the user's finger, thereby providing the similar operational features and advantages as described hereinbefore.

In sum, one aspect of the disclosed technology is directed to an endoscope comprises an insertion portion having a bendable portion being attached thereto. The bendable portion is bendable at least upwardly and downwardly. A manipulator is disposed on a proximal end side of the insertion portion and having a grip that is gripped by a user. A bend manipulating member is disposed between the insertion portion and the grip and standing up in a predetermined neutral direction when not manipulated. The bend manipulating member is tiltable toward at least the insertion portion and the grip. A finger rest which is disposed on an end portion of the bend manipulating member positioned outward from the manipulator and on which the user places a thumb thereof while gripping the grip.

An arcuate insert-side finger rest face performs as an outer surface of the finger rest on an insert side thereof and extends about a first central point and having a first radius. An arcuate grip-side finger rest face performs as an outer surface of the finger rest on a grip side thereof and extends about a second central point positioned closer to the insertion portion than the first central point and having a second radius. The first radius is longer than the second radius. The grip has a longitudinal axis whose direction crosses the neutral direction of the bend manipulating member at an angle ranging from 90 to 130 degrees. Each of the insert-side finger rest face and the grip-side finger rest face has a plurality of bumps thereon. The plurality of bumps include columnar bumps arrayed in a hexagonal shape. The plurality of bumps includes a combination of columnar bumps and dots.

A second aspect of the disclosed technology is directed to an endoscope comprises an insertion portion having a bendable portion being attached thereto. The bendable portion is bendable at least upwardly and downwardly. A manipulator is disposed on a proximal end side of the insertion portion and having a grip that is gripped by a user. A bend manipulating member is disposed on the manipulator and standing up in a predetermined neutral direction when not manipulated. The bend manipulating member is tiltable in at least a first direction and a second direction. A finger rest which is disposed on an end portion of the bend manipulating member positioned outside of the manipulator and on which the user places a thumb thereof while gripping the grip. An arcuate first finger rest face performs as an outer surface of the finger rest that extends in the first direction away from the thumb and extends about a first central point and having a first radius. An arcuate second finger rest face performs as an outer surface of the finger rest that extends in the second direction closer to the thumb than the first finger rest face and extends about a second central point positioned further in the first direction than the first central point and having a second radius.

A third aspect of the disclosed technology is directed to an endoscope comprises an insertion portion having a bendable portion. A manipulator is disposed on a proximal end side of the insertion portion and having a grip. A shaft is disposed outward from the manipulator and standing up in a neutral direction when not manipulated and is configured to be tilted toward at least the insertion portion and the grip. A finger rest is disposed on an end portion of the shaft. The finger rest having an outer surface. The outer surface includes a first surface and a second surface. The first surface is arcuate and located on the side of the insertion portion and extending about a first central point and having a first radius. The second surface is arcuate and located on the side of the grip and extending about a second central point and having a second radius. The second central point is positioned closer to the insertion portion than the first central point.

According to the present disclosure, there is realized an endoscope that includes a manipulator having an upstanding bend manipulating member that can smoothly be tilted by a finger of the hand of the user that is gripping the manipulator, placed on an outer surface of a finger contact, regardless of the size of the user's hand or the length of the user's finger.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An endoscope comprising:
an insertion portion having a bendable portion being attached thereto, the bendable portion being bendable at least upwardly and downwardly;
a manipulator disposed on a proximal end side of the insertion portion and having a grip that is gripped by a user;
a bend manipulating member disposed between the insertion portion and the grip and standing up in a predetermined neutral direction when not manipulated, the bend manipulating member being tiltable toward at least the insertion portion and the grip;
a finger rest which is disposed on an end portion of the bend manipulating member positioned outward from the manipulator and on which the user places a thumb thereof while gripping the grip;
an arcuate insert-side finger rest face performing as an outer surface of the finger rest on an insert side thereof, and extending about a first central point and having a first radius; and
an arcuate grip-side finger rest face performing as an outer surface of the finger rest on a grip side thereof, and extending about a second central point positioned closer to the insertion portion than the first central point and having a second radius.

2. The endoscope of claim 1, wherein the first radius is longer than the second radius.

3. The endoscope of claim 2, wherein the grip has a longitudinal axis whose direction crosses the neutral direction of the bend manipulating member at an angle ranging from 90 to 130 degrees.

4. The endoscope of claim 1, wherein each of the insert-side finger rest face and the grip-side finger rest face has a plurality of bumps thereon.

5. The endoscope of claim 4, wherein the plurality of bumps include columnar bumps arrayed in a hexagonal shape.

6. The endoscope of claim 4, wherein the plurality of bumps includes a combination of columnar bumps and dots.

7. An endoscope comprising:
an insertion portion having a bendable portion being attached thereto, the bendable portion being bendable at least upwardly and downwardly;
a manipulator disposed on a proximal end side of the insertion portion and having a grip that is gripped by a user;
a bend manipulating member disposed on the manipulator and standing up in a predetermined neutral direction when not manipulated, the bend manipulating member being tiltable in at least a first direction and a second direction;
a finger rest which is disposed on an end portion of the bend manipulating member positioned outside of the manipulator and on which the user places a thumb thereof while gripping the grip;
an arcuate first finger rest face serving as an outer surface of the finger rest that extends in the first direction away from the thumb, and extending about a first central point and having a first radius; and
an arcuate second finger rest face serving as an outer surface of the finger rest that extends in the second direction closer to the thumb than the first finger rest face, and extending about a second central point positioned further in the first direction than the first central point and having a second radius.

8. An endoscope comprising:
an insertion portion including a bendable portion;
a manipulator disposed on a proximal end side of the insertion portion and having a grip;
a shaft disposed outward from the manipulator and standing up in a neutral direction when not manipulated and configured to be tilted toward at least the insertion portion and the grip;
a finger rest disposed on an end portion of the shaft, the finger rest having an outer surface, the outer surface including a first surface and a second surface,
the first surface being arcuate and located on the side of the insertion portion and extending about a first central point and having a first radius,
the second surface being arcuate and located on the side of the grip and extending about a second central point and having a second radius, the second central point being positioned closer to the insertion portion than the first central point.

9. The endoscope of claim 8, wherein the first radius is longer than the second radius.
